(19) United States
(12) Patent Application Publication
(10) Pub. No.: US 2010/0124533 A1
(43) Pub. Date: May 20, 2010

(54) Large Animal Model for Human-Like Advanced Atherosclerotic Plaque

(75) Inventors: Stefan Tunev, Santa Rosa, CA (US); Ankit Shah, Santa Rosa, CA (US); Alejandra Caceres, Santa Rosa, CA (US); Ya Guo, Cotati, CA (US); Julie Trudel, Santa Rosa, CA (US); Phean Him, Santa Rosa, CA (US); Ayala Hezi-Yamit, Windsor, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 12/274,817

(22) Filed: Nov. 20, 2008

(51) Int. Cl. A61B 10/00; A01K 67/00

(52) U.S. Cl. 424/9.2; 800/9

(57) ABSTRACT

An animal model for cardiovascular disease comprising one or more vascular plaque lesions formed at selected sites within a vascular segment of a nonhuman mammal. The vascular plaque lesion is formed by administering a hypercholesterolemic diet to the nonhuman mammal, inflicting an injury to the vascular wall at the selected site after a predetermined exposure to the hypercholesterolemic diet, and applying a hydrogel to the injured vascular wall. Another aspect of the invention provides a method for evaluating a test compound for an effect on atherosclerotic lesion formation comprising administering to a nonhuman mammal a hypercholesterolemic diet, and, after a defined period of time, isolating a segment of a blood vessel using a balloon catheter, inflicting an injury to the vascular wall within the isolated segment, and applying a hydrogel within the vascular segment. The method further comprises forming a vascular plaque lesion on the vascular wall at the site of the injury, delivering the test compound to the nonhuman mammal, and monitoring atherosclerotic lesion size and composition at the injured site after a defined period of exposure to the test compound.

### TECHNICAL FIELD

This invention relates generally to an animal model of atherosclerotic cardiovascular disease wherein a vascular lesion can be induced at a preselected site. More specifically, the invention relates to a porcine model of atherosclerosis developed by deposition of at least one pro-inflammatory substance on the luminal surface of an artery in combination with a hyperlipidemic diet that results in asymmetric plaque formation having a high content of inflammatory cells and a cap-like structure.

### BACKGROUND OF THE INVENTION

Atherosclerosis, a major cause of morbidity and mortality in the United States, is a progressive disease that results in deposition of plaque on the inner lining of large and medium-sized arteries. The plaque, consisting of fatty substances including cholesterol, cellular debris and calcium, builds up slowly, and most often causes clinical symptoms beginning in middle age. The plaque may grow large enough to partially block the artery and significantly reduce blood flow to the heart and other vital organs. If blood flow to the heart is sufficiently reduced, angina (chest pain) results. However, most damage occurs when the plaque becomes unstable and ruptures, causing fragments of the plaque to break off and travel through the vasculature. These fragments then become lodged in blood vessels in other parts of the body, blocking blood flow and causing blood clots that result in further obstruction of the blood vessel. If a vessel that feeds the heart is blocked, a myocardial infarction (heart attack) may result. Similarly, blockage of an artery that supplies the brain results in a stroke; blockage of an artery within the lung results in pulmonary embolism.

Although the etiology of plaque formation is not well understood, various causal factors have been identified, including high serum cholesterol concentration, hypertension, obesity, exposure to cigarette smoke or other pollutants, and the presence of concomitant disease such as diabetes. The sensitivity of an individual to each of these factors is thought to be determined at least in part by genetic heredity.

Throughout the life of the individual, the blood vessel wall is exposed to cholesterol transported in low-density lipoprotein particles. Some of the particles enter the vessel wall and release cholesterol, which is then oxidized and initiates the inflammatory process by attracting macrophage to the site. The macrophages ingest the oxidized cholesterol and become foam cells. The foam cells and platelets that accumulate at the site continue the inflammatory process, eventually leading to the destruction of smooth muscle cells and replacing them with collagen. The collagen layer eventually extends over the fatty deposit and forms a fibrous cap between the fatty deposit and the intimal lining of the vessel. The cap may be thick, resulting in a stable plaque, or thin, resulting in an unstable plaque that is prone to rupture. Over time the artery enlarges to accommodate the growing plaque and maintain the size of the lumen. However, in some cases, the lumen of the artery eventually becomes partially blocked resulting in stenosis and reduced blood flow.

Atherosclerosis is a complex physiologic process that develops over a long period of time, making it difficult to study. Various in vitro and in vivo models have been developed to facilitate understanding and treatment of the disease. These models include cultures of isolated animal and human cells, transgenic mice, rats, rabbits, and swine. Cell culture systems can be used to determine cellular responses to various treatments, but provide little information on the in vivo process of atherosclerotic plaque formation. Transgenic mice and rats have been developed that have one or more human genes involved in lipid metabolism and develop various symptoms of atherosclerosis. Other mouse models are “knock-out” animals that have been genetically altered so that they lack one or more enzymes required for normal lipid metabolism. In either case, the arteries of these animals are small and have very thin walls compared to human arteries, thus limiting their predictive value for the treatment of human disease. Swine and other large animals such as dogs and sheep are generally preferred because the size of the heart and blood vessels more closely resembles that of humans. Among these animals, swine are considered to metabolize lipids most similarly to humans, and therefore offer a metabolic model that is predictive of human disease. However, these large animals are costly to house and maintain during the course of experiments that last for weeks or months.

It is desirable, therefore, to provide a large animal model for studying the progression and treatment of atherosclerosis that consistently forms atherosclerotic lesions in a short period of time, analogous in size and structure to human plaque. Further, it is desirable that multiple lesions that are of similar size can be formed in proximity to each other so that the safety and efficacy of novel therapies can be evaluated in a minimum number of animals.

### SUMMARY OF THE INVENTION

One aspect of the present invention provides an animal model of cardiovascular disease in which vascular plaque lesions are formed at selected sites within a vascular segment of a nonhuman mammal. The vascular plaque lesion is formed by administering a hypercholesterolemic diet to the nonhuman mammal, and, after a predetermined exposure to the hypercholesterolemic diet, inflicting an injury to the vascular wall at one or more selected sites, and applying a hydrogel to the vascular wall.

Another aspect of the invention provides a method of producing one or more atherosclerotic lesions in a nonhuman mammal by administering to the nonhuman mammal a hypercholesterolemic diet for a defined period of time. Next, after a predetermined exposure to the hypercholesterolemic diet, a segment of a blood vessel within the non-human mammal is isolated using a balloon catheter. The vascular wall within the isolated segment is injured, and a hydrogel is applied within the injured vascular segment.

Another aspect of the invention provides a method for evaluating the safety and efficacy of a test compound for an effect on atherosclerotic lesion formation in a nonhuman mammal. First, a hypercholesterolemic diet is administered to the nonhuman mammal. After exposure to the hypercholesterolemic diet for a defined period of time, a segment of a blood vessel is isolated using a balloon catheter, and an injury is inflicted on the vascular wall within the isolated segment. Next, a hydrogel is applied to the injured site within the vascular segment. Following this procedure, a vascular plaque lesion forms on the vascular wall at the site of the injury. Finally, a test compound is delivered to the nonhuman mammal. Atherosclerotic lesion size and composition at the injured site is monitored after a defined period of exposure to the test compound.

The present invention is illustrated by the accompanying figures portraying various embodiments and the detailed description given below. The figures should not be taken to limit the invention to the specific embodiments, but are for explanation and understanding. The detailed description and figures are merely illustrative of the invention rather than limiting, the scope of the invention being defined by the appended claims and equivalents thereof. The drawings are not to scale. The foregoing aspects and other attendant advantages of the present invention will become more readily appreciated by the detailed description taken in conjunction with the accompanying figures.

### BRIEF DESCRIPTION OF THE DRAWINGS

FIG. 1 is a schematic illustration of a system for creating a vascular lesion including a double balloon catheter that is designed to deliver a photo-curable macromer to discrete locations within the vascular system, in accordance with one embodiment of the present invention;

FIG. 2A is a photograph of a histological sample of a cross section of a human artery with a type IV lesion;

FIG. 2B is a photograph of a histological sample of a cross section of a porcine femoral artery treated by combination of endoluminal coating and high fat diet, day 28 post treatment, in accordance with the present invention;

FIG. 3A is a photograph of a histological sample of a cross section of a human artery showing the cell composition of a human atherosclerosis type II lesion;

FIG. 3B is a photograph of a histological sample of a cross section of a porcine femoral artery showing the cell composition of an experimental atherosclerotic lesion, in accordance with the present invention; and

FIG. 4 is a flow diagram for a method of creating vascular lesions in an experimental animal and evaluating the efficacy of therapeutic agents for treating vascular lesions, in accordance with the present invention.

### DETAILED DESCRIPTION

The present invention is directed to an animal model suitable for studying cardiovascular disease evidenced by plaque formation on vessel walls. A particular focus of the invention is an animal model that forms asymmetric plaque lesions having a high content of inflammatory cells and a fibrous cap-like structure, that are similar to those lesions observed in human cardiovascular disease that are prone to rupture and ensuing coronary thrombosis. Nonhuman mammals appropriate for the invention include rodents such as mice, rats, guinea pigs, and other small animals such as rabbits. However in some embodiments, larger animals having a vasculature similar in size and geometry to that of the human are used. In this embodiment, appropriate large nonhuman mammals are bovine, canine, ovine, porcine or primates. In one embodiment, the selected animal is porcine and is any one of Yorkshire swine, other pure-bred breeds of swine, or cross-bred swine, Yucatan minipigs, or Ossobaw pigs. Either male or female animals are appropriate for the model. In another embodiment, the experimental animals are genetically modified to attenuate or reduce the expression of one or more genes or alternatively, over-express one or more genes and, as a result, accelerate the progression of atherosclerotic disease.

In one embodiment, endocrine or metabolic changes that accelerate atherosclerotic disease or cause co-morbidities are induced in the experimental animal by modifying or removing one or more organs such as reproductive organs, liver, or pancreas. In one embodiment, a portion of the pancreas is removed, resulting in reduced insulin release and elevated serum glucose, a physiologic condition frequently accompanying atherosclerosis in human disease.

In another embodiment, pharmaceutical or biologic agents that accelerate atherosclerotic progression or induce co-morbidities are administered to the experimental animal. Examples of such agents include steroid or peptide hormones, warfarin and others.

From weaning until the initiation of the experiment, the animals are fed a hypercholesterolemic diet consisting of standardized feed that is nutritionally adequate to support normal growth, plus additional lipids. Examples of lipids that promote atherosclerosis include lard, partially hydrogenated oils, butter, saturated fatty acids, triglycerides, and cholesterol. In one embodiment, between 15 and 45% lard is added to the standardized feed. In another embodiment, between 2 and 10% cholesterol is added to the standardized feed given to the experimental animals. Simple sugars such as glucose and fructose also promote atherosclerosis, and may be added to the diet of the experimental animals. In one embodiment, experimental animals are fed a hypercholesterolemic diet comprising nutritionally adequate standardized feed, with 20% lard, 5% cholesterol, and 18% fructose added. In other embodiments, some of the added components, such as triglycerides, fructose, or glucose are administered intravenously. [00021] One aspect of the invention includes administering into the cardiovascular system of the experimental animal a hydrogel that that promotes atherosclerotic lesion formation. The hydrogel consists of an aqueous solution of one or more macromers consisting of hydrophilic polymers that make up the backbone of the polymeric structure, biodegradable polymeric segments and end groups that can be cross-linked. The hydrophilic polymers may be linear, branched, or graft polymers, and may vary in molecular weight, depending on the desired mechanical and degradation properties of the hydrogel. Suitable polymers include polyethylene oxide, polyhydroxyl methacrylate, polyvinyl alcohol, and other suitable polymers. In some embodiments the polymers include a mix of subunits or comprise block copolymers. In one embodiment, the polymers include branched polymers such as 3-arm or star-shaped polyethylene glycols.

At each end of the hydrophilic polymer, are biodegradable polymeric segments that may be either repeating units of a single monomer, or may comprise a mixture of monomers. The monomers are selected to cause the hydrogel to degrade and be removed from the treatment site within a defined period of time. In one embodiment, the hydrogel degrades within 3 to 4 weeks. Examples of suitable monomers for the degradable portion of the molecule include lactide, caprolactone, trimethylene carbonate, caprolactone derivatives, and glycolides. The biodegradable portion of the polymer varies in molecular weight, and in one embodiment is between 2 and 20 subunits.

Suitable cross-linkable end groups include any chemical group that can be cross-linked through free radical polymerization. Acrylate and methyl-methacrylate are examples of suitable chemical groups. In one embodiment, the macromer comprises a polyethylene glycol chain having a number average molecular weight of 3,350, 5 lactic acid units at each end of the polyethylene glycol chain, and an acrylate group on each end of the polymer molecule.

The hydrogel formulation is prepared by dissolving the macromer in an aqueous solution, adding a co-initiator and an accelerator, and in some cases, other additives to modulate polymerization rate. Methyl-diethanolamine, and triethanolamine are examples of co-initiators, in accordance with the invention. The accelerator is N-vinyl-caprolactam, or other highly reactive free radical monomers. The concentration of each component is adjusted to achieve the desired polymerization time for the hydrogel.

The following example illustrates preparation of a hydrogel solution, in accordance with the invention.

### EXAMPLE 1

| Materials | | |
|---|---|---|
| (500 mL Batch) | Weight (g) | |
| 3.35KL5A2 | 150.0 | |
| Water for Injection | 296.97 | |
| Biostent 10X Buffer | 50 | mL |
| n-Vinyl-Caprolactone | 2.5 | |
| Fructose | 0.5 | |
| Fe-Sulfate | 0.025 | |
| Total | 500.0 | |

Procedure:
1. Tare 1000 mL glass beaker+magnetic stir bar, record start weight.
2. Weigh 291.65 g of water for injection into beaker.
3. Weigh 0.025 g of Ferrous-sulfate heptahydrate, transfer to beaker and dissolve with stirring.
4. Weigh 0.5 g of Fructose and add to solution in beaker
5. Weigh 150.0 gram of 3,350 dalton polyethylene glycol, lactate (5 subunits), acrylate (one subunit, each end) macromer on balance, transfer to beaker, dissolve with stirring.
6. Add Biostent 10X Buffer (Genzyme, Corp., Cambridge, Mass., USA), continue stirring.
7. Add n-vinyl-caprolactone, stir until dissolved.
8. Adjust final weight of formulation to 500.0 gram if needed.

To activate the free radical cross-linking process, a photosensitive primer solution is used. The primer solution “primes” the vessel wall by coating and binding to it, so that the hydrogel, as it forms will adhere securely to the vessel wall. The primer solution contains a suitable concentration of photosensitive molecules that activate the free radical-dependent polymerization of the cross-linkable end groups of the hydrogel-forming macromers. Useful photosensitive molecules include photosensitive dyes, quinines, hydroquinones, poly-alkenes, polyaromatic compounds, ketones, unsaturated ketones, peroxides, halides, Eosin Y, Eosin B, flourone, erythrosine, flourecsein, and Indian Yellow and its' derivatives. Combinations of these photosensitive compounds are used in some embodiments. In one embodiment, the primer solution is 50 parts per million Eosin Y in lactated Ringer's solution that is sterilized by filtration before use.

The purpose of coating the injured arterial wall with the biodegradable hydrogel is to elicit inflammation and stimulate lesion formation. In one embodiment, the hydrogel is also used to deliver a biologically active compound that will accelerate the formation of an atherosclerotic lesion at an injured site. Compounds that may be incorporated into the hydrogel, delivered to the injured site and released over a defined period of time include pro-inflammatory drugs, and pro-apoptotic cytokines and chemokines such as TNFα, CD-40 ligand, interleukin-1β, interleukin-8, interleukin-6; pro-thrombotic and pro-coagulatory molecules such as coagulation Factor VIIa, Factor Xa, thrombin, molecules that activate platelets, such as PAR-1 and PAR-4 agonists, and collagen; pharmaceutical agents that induce cell death, toxicity or inflammation, for example Staurosporin; bioactive molecules that induce macrophage apoptosis, or lipid accumulation and, as a result, accelerate atherosclerosis; bacterial or viral derivatives such as cell wall lipopolysaccharides (LPS) that induce toll-like receptor (TLR) signaling, and agonists and ligands that induce activation of TLR-2 and TLR-4 receptors; and biological molecules, enzymes and chemicals that induce oxidative stress at the plaque site. The composition of the hydrogel and the concentration of one or more these compounds are selected to produce a vascular lesion at the treatment site within approximately 28 days post treatment.

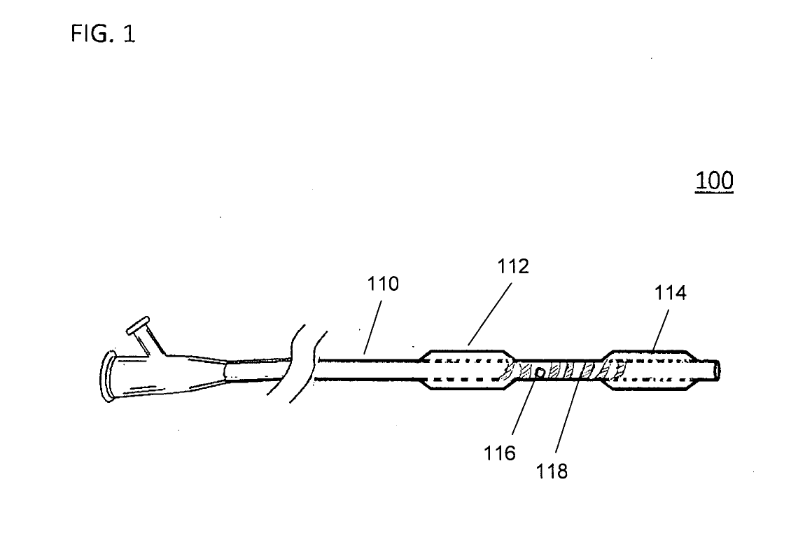

FIG. 1 is an illustration of a system 100 for creating an atherosclerotic vascular plaque lesion, comprising a catheter 110 that is designed to deliver a photo-curable macromer to a discrete location of the vascular anatomy. In an exemplary embodiment, catheter 110 includes two expandable balloons, 112 and 114, that can be inflated separately by pressurizing a fluid such as contrast fluid or saline solution that flows through a lumen connected to the respective balloon. Catheter 110 further comprises an internal solution delivery sheath or lumen, having an orifice 116 between balloons 112 and 114, and a fiber optic diffuser device 118, located under and between the balloons.

Fiber optic diffuser device 118 is connected to a Diode Pumped Solid State (DPSS) laser having a continuous output of 532 nm wavelength. A standard 120 volt AC power outlet is used to supply power to the DPSS laser. Output power is variable between 0 and 2 watts, maximum. Light diffuser device 118 delivers between 280 and 340 milliwatts/cm²of energy density to the vessel wall.

To create the lesion, the distal portion of the catheter is advanced over a 0.014 inch guide wire through the vascular system until distal balloon 114 is located at the site selected for the vascular lesion. Next, distal balloon 114 is inflated repeatedly so that the vessel wall is stretched sufficiently to cause injury to the wall. In one embodiment, distal balloon is inflated three times for 60 second time periods, stretching the vessel wall so that the diameter of the vessel lumen is increased by 30%. Between each inflation, balloon 114 is moved back and forth longitudinally within the vessel so that the endothelial layer of the vascular wall is abraded and removed.

After the injury to the vessel wall has been created, double-balloon catheter system 100 is advanced so that the injured site of the vessel wall is placed between balloons 112 and 114. Both balloons 112 and 114 are inflated so that blood flow is occluded, but fluid can flow from the chamber formed by the two balloons over the surface of balloon 114. The portion of the artery between balloons 112 and 114 is then flushed with approximately 5.0 to 10 ml lactated Ringer's saline solution to remove excess blood. Next, the pressure in balloon 114 is adjusted so that the chamber between balloons 112 and 114 is tightly sealed isolating the vascular segment surrounding the injured site. Approximately 5.0 ml of a primer solution and 5.0 ml of lactated Ringer's solution are injected into the chamber. Next, 5.0 ml of macromer solution is delivered to the chamber, and illuminated with 532 nm wavelength laser energy from light diffuser 118 for 20 seconds, causing in situ photo-polymerization of the macromer and formation of a hydrogel within the injured vessel segment. The balloons are then deflated, and the catheter removed from the vasculature. Nitroglycerine or other vasodilators are administered to the animal to control vasospasm, if needed.

In one embodiment the presence of the hydrogel causes formation of an atherosclerotic plaque lesion at the injured site on the vessel wall. In another embodiment, a pro-inflammatory agent is incorporated into the macromer solution and delivered into the chamber. Following treatment, the pro-inflammatory agent is released at the treatment site, further promoting atherosclerotic lesion formation. In either embodiment, over a period of two to three weeks, the hydrogel degrades and is removed from the treatment site.

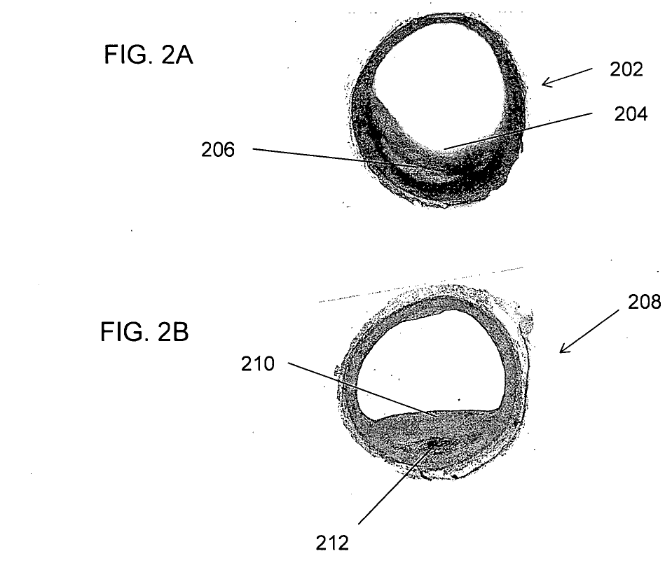

FIG. 2B is a cross section 208 of a porcine femoral artery treated by a combination of endoluminal coating with a hydrogel and a high fat diet, at 28 days post treatment. The internal diameter of the artery is narrowed due to the presence of atherosclerotic plaque 210. Histological evaluation of the vascular tissue at the treatment site indicates eccentric pale yellow neointimal tissue buildup that results in mild to moderate reduction of vascular lumen. The histomorphological composition of this neointimal reaction is consistently observed at all treated vascular sites, and is characterized by superficial areas composed of smooth muscle cells and extracellular-matrix, that form a cap-like structure over the surface of the lesion, and deep areas occupied by inflammatory cells including lipid laden (foamy) macrophages 212. The foamy macrophages have an eccentric nucleus and increased cytoplasmic space filled with small, sharply demarcated and clear vacuoles (fatty vacuoles). Similar fatty vacuoles are occasionally present within adjacent smooth muscle cells. The internal elastic lamina and tunica media is histologically intact. These lesions are grossly and microscopically very similar to the human atherosclerosis types II (fatty streak) and III (intermediate) lesions, (Atlas of Atherosclerosis, Herbert C. Stary, ed., Second Edition, 2003). For comparison, FIG. 2A is a cross section is a human anterior descending coronary artery 202 with a type IV lesion 204 with areas of foamy macrophage 206. Both the human and porcine lesions exhibit reduction of the vascular lumen that is characteristic of atherosclerotic plaque lesions.

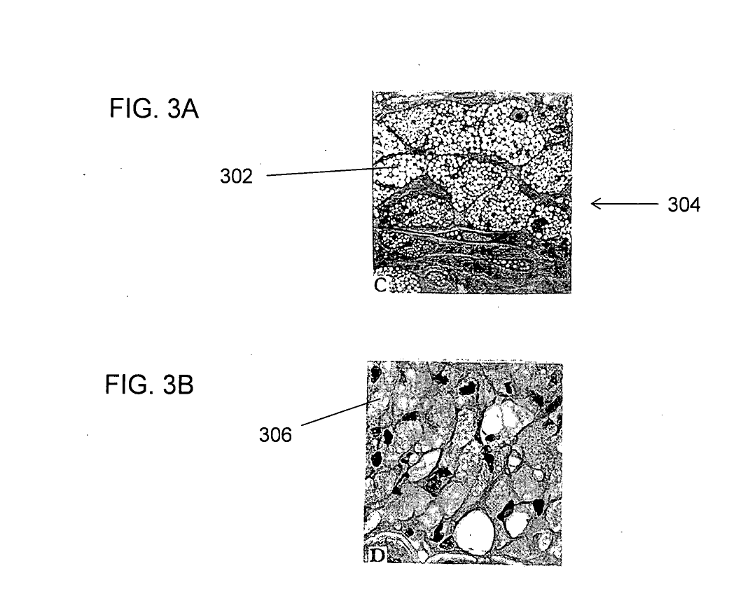

FIG. 3A is a histological preparation showing the cell composition of a human atherosclerotic type II lesion that formed in the anterior descending coronary artery. Foamy macrophage 302 are inflammatory cells, and are widespread in the upper intima 304. FIG. 3B is a histological preparation showing the cellular composition of a lesion occurring in an artery of the porcine experimental model for atherosclerosis. In this specimen, the deeper portion of the neointima is occupied by tightly packed inflammatory cells, especially foamy macrophages 306.

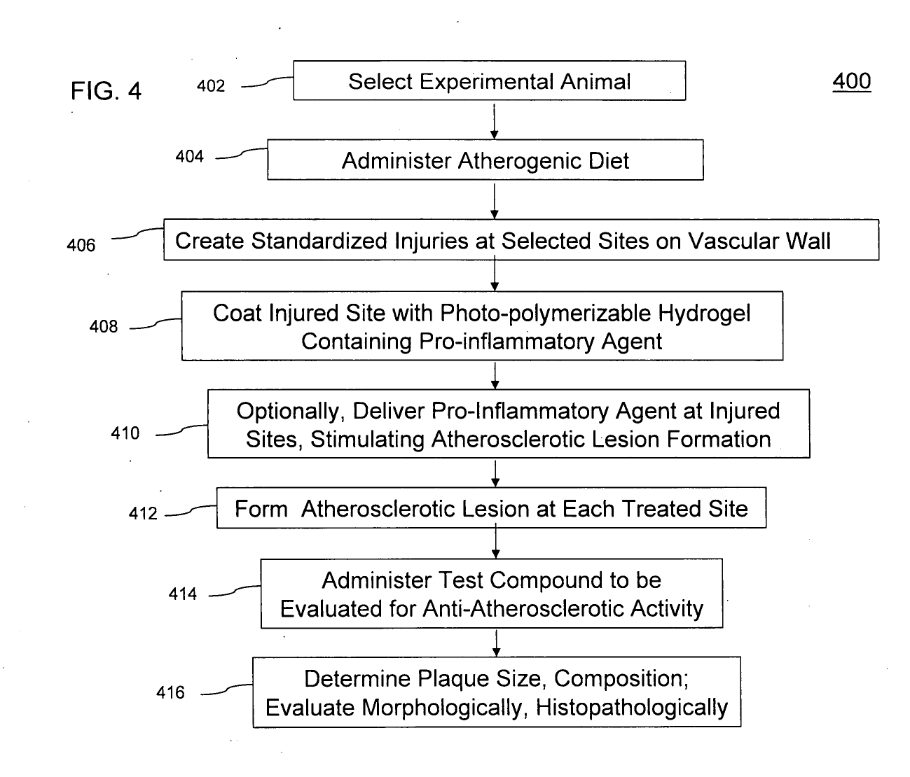

FIG. 4 is a flowchart of method 400 for evaluating the efficacy of a therapeutic agent for an effect on atherosclerotic lesion formation in an animal model for human atherosclerotic disease. The method includes first, selecting an appropriate animal, as indicated in Block 402. In one embodiment, Yorkshire swine are selected. The swine are maintained on a hypercholesterolemic diet consisting of standardized pig chow supplemented with 20% lard, 5% cholesterol and 18% fructose for a defined period of time, for example, until they are at least 9 months of age, as indicated in Block 404. Between 9 and 12 months of age, the pigs are weighed and their serum cholesterol is measured regularly. When the animals weigh between 35 and 65 kilograms, and their serum cholesterol is at least 100 mg/dL, they are subjected to vascular injury (Block 206), and gel deposition (Block 408) at the selected sites.

To form lesions, sites are selected in the femoral artery, or other large artery. The pig is anesthetized, and a double balloon catheter system 100 is advanced through the vascular system until distal balloon 114 of catheter 110 is adjacent the site selected for lesion formation. Balloon 114 is then inflated three times for 60 second time periods, stretching the vessel wall so that its' inner diameter is enlarged by approximately 30%. Between the balloon inflations, flaccid balloon 114 is rubbed over the injured site abrading the endothelial cell layer from the vessel wall. This process is repeated at multiple sites in the arterial vasculature.

Next, the catheter is positioned at each injured site so that the injured vessel wall is positioned between balloons 112 and 114. Both balloons 112 and 114 are inflated so that a tight chamber is formed and creates an isolated vascular segment that includes the injured site, and a primer solution, diluted with lactated Ringer's saline solution is injected into the chamber. The primer solution contains a photosensitive molecule such as Eosin Y. The liquid macromer solution is then injected into the sealed chamber and allowed to mix with the primer solution. Optionally, the macromer solution may contain a pro-inflammatory compound that will accelerate lesion formation. The macromer comprises a hydrophilic polymeric backbone with biodegradable portions and photo-sensitive end groups. A laser light is conducted through a fiber optic wire in the catheter and diffused into the chamber. The laser light is absorbed by the photo-sensitive primer, which in turn activates the free radical-dependent polymerization of the cross-linkable end groups and, causes chemical cross-linking of the macromer molecules, and formation of a viscous hydrogel in the chamber. Finally, balloons 112 and 114 are deflated, and catheter 100 is removed from the vascular system, leaving an injury to the vessel wall coated or paved with a hydrogel containing a pro-inflammatory agent at each site.

During a time period of several days or weeks, the pro-inflammatory agent, if present, is delivered from the hydrogel to the injured site on the vessel wall, stimulating atherosclerotic plaque formation (Block 410). In addition, the hydrogel degrades, and is removed from the site. After about 28 days, an atherosclerotic lesion is formed at each treated site, and indicated in Block 412.

Next, as indicated in Block 414, the animal is treated with one or more test compounds to be evaluated for an effect on atherosclerosis. The test compound may be administered orally, intravenously, or by any other means, for example dietary manipulation. After a suitable time period, the animal is sacrificed and the atherosclerotic lesion sites are evaluated morphologically and histologically for changes in plaque size and composition, as indicated in Block 416.

While the invention has been described with reference to particular embodiments, it will be understood by one skilled in the art that variations and modifications may be made in form and detail without departing from the spirit and scope of the invention.

1. An animal model for cardiovascular disease, the model comprising:
at least one vascular plaque lesion, formed at a selected site within a vascular segment in a nonhuman mammal, the vascular plaque lesion formed by:
administering a hypercholesterolemic diet to the nonhuman mammal;
inflicting an injury to the vascular wall at the selected site wherein the injury is inflicted after a predetermined exposure the hypercholesterolemic diet; and
applying a hydrogel to the injured vascular wall.

2. The animal model of claim 1 wherein the nonhuman mammal is bovine, canine, ovine, porcine or primate.

3. The animal model of claim 2 wherein the nonhuman mammal is porcine, and is selected from the group consisting of Yorkshire swine, Yucatan minipigs, Ossobaw pigs, other breeds of swine, and cross-bred swine.

4. The animal model of claim 1 wherein the hydrogel includes at least one macromer, the macromer comprising a hydrophilic polymer having biodegradable subunits attached to at least one end of the hydrophilic polymer, and cross-linkable end groups on each end of the macromer.

5. The animal model of claim 4 wherein at least one hydrophilic polymer is photo-polymerizable.

6. The animal model of claim 5 further comprising:
applying a photosensitive primer solution to the vascular wall within a vascular segment; and
forming the hydrogel in situ by photo-polymerization within the vascular segment adjacent the injured vascular wall.

7. The animal model of claim 1 wherein at least one biologically active compound is delivered to the vascular wall at the injured site to induce at least one of cell death, toxicity, inflammation, macrophage apoptosis, lipid accumulation, thrombosis, and oxidative stress at the injured site within the vascular segment.

8. The animal model of claim 1 wherein the vascular plaque lesion is an asymmetric plaque formation with a high content of inflammatory cells and a fibrous cap-like structure.

9. A method of producing at least one atherosclerotic lesion in a nonhuman mammal comprising:
administering to the nonhuman mammal a hypercholesterolemic diet;
isolating a segment of a blood vessel within the non-human mammal via balloon catheter after a predetermined exposure to the hypercholesterolemic diet;
inflicting an injury to the vascular wall within the isolated segment of the blood vessel; and
applying a hydrogel within the isolated vascular segment.

10. The method of claim 9 wherein the nonhuman mammal is bovine, canine, ovine, porcine or primate.

11. The method of claim 9 wherein the hydrogel includes at least one macromer, the macromer comprising a hydrophilic polymer having biodegradable subunits attached to at least one end of the hydrophilic polymer, and cross-linkable end groups on each end of the macromer.

12. The method of claim 9 wherein at least one hydrophilic polymer is photo-polymerizable.

13. The method of claim 12 further comprising:
applying a photosensitive primer solution to the vascular wall within a vascular segment; and
forming the hydrogel in situ by photo-polymerization within the vascular segment adjacent the injured vascular wall.

14. The method of claim 9 further comprising:
delivering at least one biologically active compound to the vascular wall at the injured site to induce at least one of cell death, toxicity, inflammation, macrophage apoptosis, lipid accumulation, thrombosis, and oxidative stress within the injured vascular segment.

15. The method of claim 9 further comprising;
forming a vascular plaque lesion at the injured site of the vascular wall that is an asymmetric plaque formation having a high content of inflammatory cells and a fibrous cap-like structure.

16. A method for evaluating a test compound for an effect on atherosclerotic lesion formation in a non-human mammal comprising:
administering to the nonhuman mammal a hypercholesterolemic diet;
isolating a segment of a blood vessel within the nonhuman mammal via balloon catheter;
inflicting an injury to the vascular wall within the isolated segment after exposure to the hypercholesterolemic diet for a defined period of time;
applying a hydrogel within the vascular segment;
forming a vascular plaque lesion on the vascular wall at the site of the injury;
delivering the test compound to the nonhuman mammal; and
monitoring atherosclerotic lesion size and composition at the injured site after a defined period of exposure to the test compound.

17. The method of claim 16 further comprising:
forming a vascular plaque lesion at the injured site of the vascular wall that is an asymmetric plaque formation having a high content of inflammatory cells and a fibrous cap-like structure.

18. The method of claim 17 wherein the composition of at least one atherosclerotic lesion is changed.

19. The method of claim 16 further comprising:
delivering a biologically active compound to the vascular wall at the injured site and to induce at least one of cell death, toxicity, inflammation, macrophage apoptosis, lipid accumulation, thrombosis, and oxidative stress within the injured vascular segment.

20. The device of claim 16 further comprising:
applying a photosensitive primer solution to the vascular wall within a vascular segment; and
forming the hydrogel in situ by photo-polymerization within the vascular segment adjacent the injured vascular wall.
